(19) United States
(12) Patent Application Publication
(10) Pub. No.: US 2012/0053673 A1
(43) Pub. Date: Mar. 1, 2012

(54) METHOD AND SYSTEM OF DEVICES FOR PERMANENT ACCESS TO THE CIRCULATORY SYSTEM FOR CHRONIC HEMODIALYSIS

(75) Inventors: Arthur L. Golding, Los Angeles, CA (US); Daniel Allen Cota, San Diego, CA (US); Eric Warner, Oceanside, CA (US)

(73) Assignee: IMTEC BIOMEDICAL INC., San Diego, CA (US)

(21) Appl. No.: 13/215,843

(22) Filed: Aug. 23, 2011

Related U.S. Application Data
(60) Provisional application No. 61/376,140, filed on Aug. 23, 2010.

(51) Int. Cl. A61M 25/04; A61F 2/82

(52) U.S. Cl. 623/1.15; 604/175

(57) ABSTRACT

The system and method provide ways to achieve vascular access, e.g., for chronic hemodialysis. The system includes a port which may be bonded to a vascular graft which is installed between an artery and vein. A movable seal occludes a lumen of the port which when deployed allows access to the blood flow, allowing hemodialysis. A stent may be employed with an extension that is part of the graft. A connector may lock on to the port to deploy the seal to make a connection between the patient and a dialyzer. A cap may cover the port and seal for sterility. Methods of using the system are also disclosed.

### CROSS-REFERENCE TO RELATED APPLICATIONS

This application claims benefit of priority of U.S. Provisional Patent Application Ser. No. 61/376,140, filed Aug. 23, 2010, entitled “A METHOD AND SYSTEM OF DEVICES FOR PERMANENT ACCESS TO THE CIRCULATORY SYSTEM FOR CHRONIC HEMODIALYSIS”, and is related to U.S. patent application Ser. No. 12/555,608, filed Sep. 8, 2009, entitled “METHODS AND APPARATUS FOR VASCULAR ACCESS”, both owned by the assignee of the present invention and herein incorporated by reference in their entireties.

### FIELD OF THE INVENTION

The invention relates to Hemodialysis, and more particularly to Methods and a System of Devices for Permanent Access to the Vascular System for performing Hemodialysis.

### BACKGROUND

There are 20 million patients worldwide who suffer from Chronic Kidney Disease (CKD). The majority of these patients will progress to the complete loss of kidney function defined as End Stage Renal Disease (ESRD). Patients with ESRD require dialysis to sustain their life. Dialysis removes the excess body fluids and toxic waste products of metabolism that accumulate in the patient with kidney failure. The incidence of ESRD is rapidly increasing due to many factors including aging of the population, diabetes, hypertension, arteriosclerosis, infection, drugs and chemical damage. It is estimated that worldwide over 3 million patients have ESRD requiring treatment.

Hemodialysis (HD) is the method of dialysis used by over 90% of ESRD patients. HD involves a continuous exchange of the patient's blood at high flow rates between the patient's blood vessels and the dialysis machine (Dialyzer). The excess fluid and toxic waste products are removed from the patient's blood as it passes through the Dialyzer. Treatments must be done at a minimum every other day (three times each week) for a period of 4 hours per treatment.

Creating and maintaining Vascular Access, permanent access to the patient's blood vessels, for Chronic Hemodialysis, is a major problem for ESRD patients and their physicians. It requires imaging studies, complex surgical and interventional radiologic procedures and repeated hospitalizations.

### Current Methods for Vascular Access

At present three surgical techniques are used for creating VASCULAR ACCESS for HD: Autogenous ArterioVenous Fistula (AAVF); Graft ArterioVenous Fistula (GAVF); Central Venous Catheter (CVC).

These techniques have been used, in principle, for over 30 years. Each has significant problems associated with its use and all have high rates of failure over time.

The AAVF is constructed by anastomosing (joining) a suitable vein to an adjacent artery. The flow of arterial blood into the vein causes it to increase in diameter, develop a thickened wall, become easily visible and palpable beneath the skin and carry blood at high flow rates. The vein must “mature” (increase in diameter and develop a thickened wall) for several months before it can be used safely for VASCULAR ACCESS. Unfortunately, less than 60% of dialysis patients have adequate veins for creating an AAVF and many patients require complex vein transpositions (relocations) to produce a satisfactory AAVF for VASCULAR ACCESS.

The GAVF is a “variation” of the AVF that uses a vascular graft of polytetrafluroethylene (PTFE) as a substitute for a suitable vein. The graft (measuring approximately 40 cm in length by 6-7 mm in diameter) is anastomosed to an artery, tunneled directly beneath the skin for a considerable distance and then anastomosed to an available vein. The graft, due to its size and superficial position, is easily visible and palpable and carries blood at high flow rates. It takes several weeks for the tissues surrounding the graft to become adherent to its outer surface before it can be safely used for VASCULAR ACCESS.

The CVC is a tube composed of various materials (depending on the manufacturer) 5-10 mm in diameter containing two (2) lumina (channels). It is inserted percutaneously (through the skin using a needle) into a major vein, usually the internal jugular vein, in the neck. A portion of the device may be tunneled under the skin for several cm. before exiting the skin. The external portion of the CVC consists of a pair of connectors, one for each lumen, that provide a means of connecting to the Dialyzer to provide a continuous flow of blood between patient and Dialyzer. The CVC can be used immediately after insertion but it is a temporary technique for VASCULAR ACCESS due its high incidence of infection, thrombosis (clotting) and damage to the central vein into which it has been inserted.

In order to initiate a HD treatment using either an AAVF or a GAVF, two (2) large diameter needles are inserted through the skin and tissues overlying the patient's AAVF or GAVF. The needles then must carefully puncture the wall of the vein (AAVF) or graft (GAVF) and be satisfactorily positioned within the lumen in order to obtain adequate blood flow for the continuous exchange of blood between patient and Dialyzer.

The repeated needle punctures (over 300 each year) required for HD can produce multiple complications including: 1) damage to the vein or graft wall resulting in stenosis (narrowing) of the lumen and intraluminal thrombus formation (clot), 2) hematoma (leakage of blood into the tissues), 3) false aneurysm formation (large blood filled spaces in the tissues that communicate thru defects in the Graft wall with the blood flow within the graft), 4) true aneurysms (massive enlargement of the vein lumen).

These complications can result in progressive reduction of blood flow and eventual thrombosis of the AVF or GAVF.

Difficult needle placement can result in external bleeding or inadequate blood flow for HD.

Patient movement is very limited during HD due to the danger of inadvertent needle displacement or dislodgement.

Patients with ESRD have a reduced ability to form clots and stop bleeding (hemostasis). When needles are removed at the completion of dialysis pressure must be applied to the site of removal for 10-30 minutes to produce satisfactory hemostasis and coaptation of the tissue at the site of needle puncture to prevent late bleeding.

The risk of local and systemic (blood borne) infection is increased due to the skin and tissue damage and the potential introduction of bacteria with each needle puncture despite careful cleansing of the skin. This is a particularly serious problem with GAVF due to the “foreign material” present which once infected has to be removed.

Patients suffer the pain of repeated needle punctures and the anxiety related to improper needle insertion, inadequate blood flow for HD and the possibility of late bleeding. The extremity where the access is located becomes unsightly due to the needle punctures, skin damage, hematomas, true and false aneurysms, enlarged veins and surgical scars that invariably result from VASCULAR ACCESS requiring the use of needles.

Approximately forty percent of ESRD patients do not have adequate veins for an AAVF and require a GAVF for chronic HD. The GAVF to vein anastomosis and the segment of vein that receives the blood flow from the GAVF are termed the Venous Outflow Tract (VOT). The VOT of the GAVF becomes stenotic due to: the formation of abnormal tissues originating from the vein wall, termed neointimal hyperplasia (NIH), the hypertrophy of the vein wall, termed venous remodeling (VR), and the effects of cellular elements and proteins from the blood stream. Venous Stenosis (VS) occurs and progresses within months following construction of the GAVF and results in reduced blood flow rates and eventual thrombosis of the GAVF.

Transcutaneous devices (devices traversing the skin from the external environment to the deep tissues) have the risk of local infection at the skin entrance/exit site. Despite meticulous local care and the use of various materials placed on, or modifications of, the device surface to promote tissue ingrowth, and inhibit bacterial growth and biofilm formation, minimal success has been achieved in preventing infection. The materials used and the surface modifications have not resulted in the induction of a well vascularized tissue ingrowth capable of presenting a physiological and anatomical impediment to infection.

### SUMMARY OF THE INVENTION

In certain implementations, the System may include:
1) a titanium port, of specified design, with an external surface having a silicone material, e.g., a proprietary one, to induce tissue ingrowth;
2) a vascular graft composed of polytetrafluroethylene, of specified configuration, having a section of increased diameter from which a right angled branch originates and is bonded to the titanium port by a technique, which may be proprietary;
3) a polytetrafluroethylene covered expandable stent with an angled tubular extension that may be joined to or is an integral part of the vascular graft;
4) a connector, of specified design, that by a specified mechanism, locks on to and is released from the port, that accesses the channels within the seal and automates and controls the vertical movements of the seal so as to deploy the seal channels within the graft lumen and retract the seal to its nondeployed position within the port lumen; and
5) a seal tool, of specified design, that can remove the seal from the port lumen and replace it with a new sterile seal from within its housing.

The titanium port:
1) may be implanted in the deep tissues and exit thru the skin;
2) may have an external surface coating that induces tissue ingrowth that prevents or reduces the incidence of local infection;
3) may have an exposed lip that provides an asymmetric locking mechanism that when interfaced with the connector locking mechanism aligns the connector channels with the seal channels in a configuration that conducts blood flow to the appropriate inflow and outflow tubing of the dialyzer;
4) may have a luminal surface that:
   a) provides a set of grooves that in conjunction with the design of the seal provide a method of controlling the movements of the seal and securing the seal within the port lumen; and
   b) aids the fixation of the drive shaft lifter hooks to the seal lift tabs for providing upward movement of the seal at the termination of dialysis;
5) may have a configuration at the inferior luminal orifice that provides a blood tight junction with the seal that prevents blood from entering the port lumen at all times;
6) may have, in the nondeployed position, a configuration that presents, in conjunction with the seal and graft, a smooth nonthrombogenic surface to the flow of blood within the graft;
7) may have a perforated flange that extends from the port's external surface and provides a method of fixing the port to the deep tissues and stabilizing the port for immediate use of the System following surgical implantation.

The polytetrafluroethylene graft:
1) may include a section of increased diameter to provide sufficient cross sectional area to:
   a) allow deployment of the seal within its lumen;
   b) prevent contact between seal and graft;
   c) provide high blood flow volumes for:
      i. hemodialysis;
      ii. prevention of thrombus formation; and
      iii. prevention of recirculation of blood returned from the dialyzer;
2) may be attached to the port by a right angled branch of the graft which is circumferentially wrapped and thermally bonded to the external surface of the port;
3) may be joined to the outflow vein using a polytetrafluroethylene covered expandable stent, either forming an integral part of the graft structure or joined to it secondarily, that reduces/prevents stenosis of the venous outflow tract.

The cap:
1) may contain within the concavity of its dome shaped inner surface a compressible material that contains an antimicrobial solution;
2) when positioned on the port, may elute an antimicrobial solution that bathes the port and seal surfaces; and
3) may lock on to and release from the port using a self-locking mechanism.

The seal:
1) may have channels with a conical shaped upper section with dimensions that result in a secure, fluid tight, smooth luminal junction between a channel insert positioned in the upper section and the lumen of the lower channel section;
2) may have the channel openings of the lower sections positioned, at 180 degrees, on the seal circumference to maximize the separation of inflow and outflow blood streams when the seal is deployed within the graft lumen to minimize recirculation; and
3) may have a pair of lateral tabs protruding from the external surface of the seal and seated within a set of grooves on the luminal surface of the port controlling and limiting seal movement within the port lumen.

The connector:
1) may be constructed of component parts whose coordination and actions are automated by the rotation of a control knob. These actions may provide:
   a) rapid, sterile locking of the connector on the port;
   b) rapid release of the connector from the port;
   c) prevention of removal of the seal when connector is released;
   d) deployment of the seal within the port lumen to initiate blood flow for hemodialysis; and
   e) return of the seal to its secured position within the port at the termination of dialysis.
2) may have a control knob whose rotations are as follows:
   a) clockwise rotation of the Control Knob induces downward vertical movement of the integrated components of the connector deploying the seal within the graft lumen; and
   b) counter-clockwise rotation of the knob induces upward vertical movement of the integrated components of the connector returning the seal and its channel openings to within the port lumen and reconfiguring the smooth, nonthrombogenic blood interface between port, seal, graft and blood stream;
3) may align the channels within its component parts with the seal channels and the external blood tubing of the dialyzer to transport arterial inflow blood to the dialyzer and dialyzed blood to the venous outflow tract of the graft;
4) may be sterile, disposable, light weight and small; and
5) may require minimal training, dexterity and skill to use.

### BRIEF DESCRIPTION OF THE DRAWINGS

FIG. 1 illustrates a system including an exemplary connector locked onto an exemplary port according to an embodiment of the current invention.

FIG. 2 illustrates an implant including an exemplary port on a graft according to an embodiment of the current invention.

FIG. 3 illustrates an exemplary cap according to an embodiment of the current invention.

FIG. 4 illustrates an exemplary seal, a component of the port, according to an embodiment of the current invention.

FIG. 5 illustrates an exemplary graft, a component of the port, according to an embodiment of the current invention.

FIG. 6 illustrates an exemplary connector according to an embodiment of the current invention.

FIG. 7A illustrates an exemplary port in cross section according to an embodiment of the current invention.

FIG. 7B illustrates an exemplary nondeployed port and seal in cross section according to embodiment of the current invention.

FIG. 8 illustrates an exemplary connector body according to an embodiment of the current invention.

FIG. 9 illustrates an exemplary connector control knob according to an embodiment of the current invention.

FIG. 10 illustrates an exemplary connector driveshaft according to an embodiment of the current invention.

FIG. 11 illustrates an exemplary connector channel inserter according to an embodiment of the current invention.

FIG. 12A illustrates an exemplary connector clamp according to an embodiment of the current invention.

FIG. 12B illustrates an exemplary connector clamp, channel inserter, and driveshaft, according to an embodiment of the current invention.

FIG. 13 illustrates an exemplary connector joined to an exemplary port according to an embodiment of the current invention, with the seal in a nondeployed configuration.

FIG. 14 illustrates system for dialysis including an exemplary connector joined to an exemplary port according to an embodiment of the current invention, with the seal in a deployed configuration.

### DETAILED DESCRIPTION

The purpose of the method and system of devices described is to achieve one or more of the following goals for VASCULAR ACCESS for Chronic Hemodialysis:
1) substantially eliminate the use of needles and their multiple associated problems,
2) substantially prevent or reduce the incidence of venous outflow tract (VOT) stenosis and the associated graft thrombosis,
3) substantially minimize the occurrence of exit/entrance site infections and recirculation,
4) provide a substantially permanent, high volume blood flow system for chronic hemodialysis,
5) enable substantially immediate use following surgical implantation if emergency dialysis indicated,
6) provide a system of VASCULAR ACCESS that does not require technical skills or manual dexterity for its use,
7) enable care providers at substantially all levels of training, and motivated patients, to use the system, by substantially automating the connection of the patient's circulation to the dialyzer,
8) substantially reduce the need for repeated radiological and surgical procedures to maintain functional VASCULAR ACCESS for HD.

One or more of these goals may be achieved by, in one embodiment, performing the following steps:
1) Implanting a permanent, small diameter, low-profile, transcutaneous, titanium Port (FIG. 7).
2) Covering the external surface of the implanted Port with a silicone-based porous material, which in some cases is proprietary, that induces well-vascularized tissue ingrowth minimizing the risk of local infection (16).
3) Bonding to the Port a polytetrafluroethylene (PTFE) Vascular Graft (FIG. 5, label 35), that is anastomosed to an artery and joined to a vein to create a GAVF as the source of blood flow for HD.

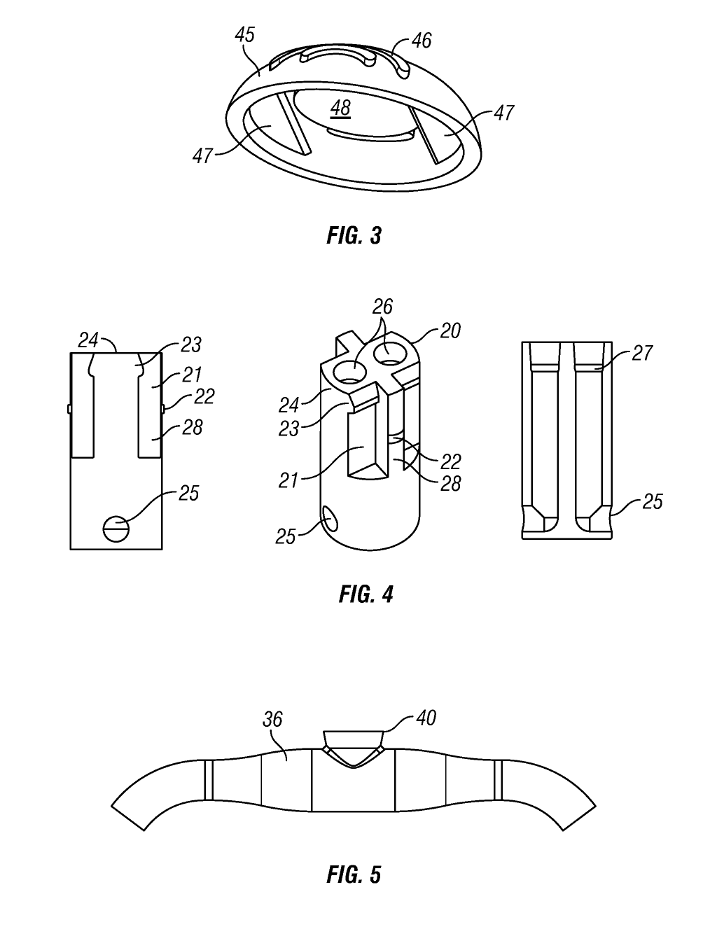

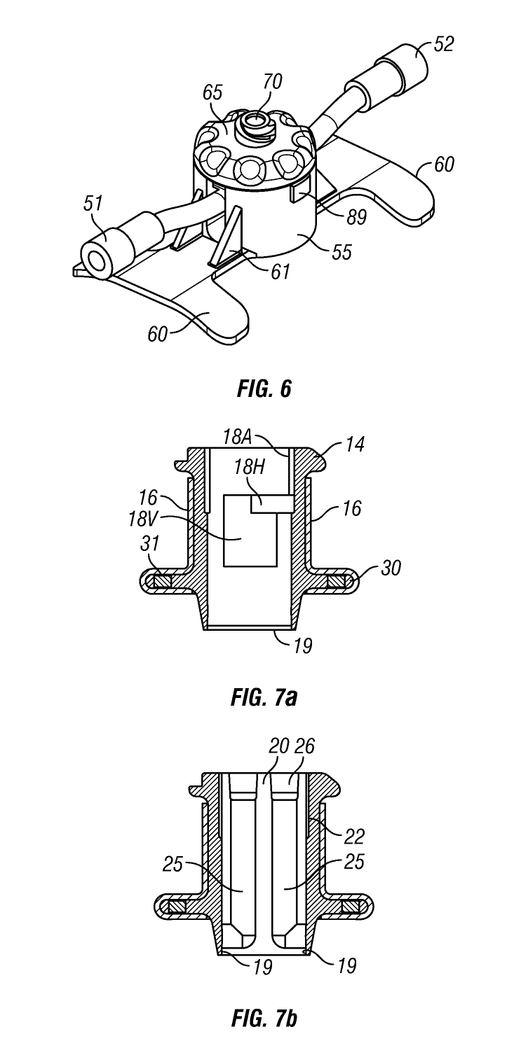

4) Occluding the lumen of the Port with a moveable Seal (FIGS. 4, 7) that contains Seal Channels (25) for transporting blood flow to and from the patient during dialysis. The Seal is in the “deployed” position when it is partially inserted into the Graft lumen, opening its channels for blood flow; and it is in the “nondeployed” position when it is within the Port lumen, closing its channels to blood flow. The Seal prevents entry of blood into the Port lumen at all times and can be removed and replaced using a specific tool if required.
5) Using a, in some cases proprietary, PTFE-covered, expandable Stent with an extension joined to, or an integral part of, the Graft and placed within the lumen of the venous outflow tract to reduce the development of VOT stenosis.
6) Providing a sterile, disposable, automated Connector (FIG. 6), that locks on to the Port and by its activation deploys the Seal Channel Openings (25) into the Graft lumen to access blood flow (FIG. 14); this may allow the simultaneous and continuous flow of blood between patient and Dialyzer. At the completion of dialysis, the action of the Connector returns the Seal to its secured position within the Port lumen.
7) Covering the Port and Seal with a sterile, disposable, antimicrobial eluting Cap (FIG. 3), that is locked on to the port when not in use for HD.

It will be understood that the descriptions above and below are exemplary in nature and that other embodiments will likewise be encompassed by the scope of the invention.

### Port (FIG. 7A)

The Port may be a thin-walled titanium cylinder which may be of various heights and diameters and may have a central lumen of various diameters. A Port Lip (14) may extend outward from the superior rim of the Port a variable distance and may contain an asymmetric set of Port Connector Grooves (17) that may function to secure a Connector (described below) in a specific orientation when the Port is in use for HD or a Cap (described below) when not in use for HD. The Port wall may vary in thickness. A moveable cylindrical Seal (described below) may occlude the Port lumen at all times. However, by means of an inferior (downward) vertical movement, Channels within the Seal may be deployed for HD within the blood stream flowing through the Graft that is bonded to the Port. The Port may have at its inferior rim including the distal 1.0 mm. of the surface of its lumen, as an integral part of the Port wall, an intraluminal circumferential protrusion of titanium so configured and of such dimensions as to function as an “O” ring of titanium (19). This “O” ring, in conjunction with the Seal, provides a fluid tight junction to prevent blood from the Graft lumen entering the Port lumen at all times whether the Seal is in the deployed position (channel openings within Graft lumen) or nondeployed position (channel openings within the Port lumen).

A titanium Flange (30), which may be an integral part of the Port wall, may extend a variable distance circumferentially from the external surface of the Port, at various distances from the superior rim of the Port. The Flange may be of minimal thickness and may contain a plurality of perforations which may be used for placement of sutures or other devices to fix the Port to the surrounding tissues and prevent the Port's movement (31).

The titanium comprising the external surface of the Port may have a roughened surface and a series of superficial circumferential grooves to enhance the adherence of various materials that may be bonded to it such as: 1) the proprietary “Star Sprinkle System” available from Healionics Corporation of Seattle, Wash., composed of silicone and 2) PTFE, of various configurations, porosities, laminations and dimensions, available from multiple manufacturers.

The inner luminal surface of the Port may have two sets of Grooves positioned on opposing walls, each set may include three connected Grooves; a Horizontal Groove (18 H) extending for 90 degrees of the Port's luminal circumference, a wide Descending Vertical Groove (18 V) of sufficient length that it may control and limit the vertical movements of the Seal when initiating and terminating HD (described below) and an Ascending Vertical Groove (18A) that may continue to the superior rim of the Port. The Horizontal and Ascending Grooves may allow the Seal to be rotated within the Port lumen and be retracted upward for removal and by reverse movements be replaced with a new sterile Seal, without blood loss, when a Seal Tool of special design is used (described below).

### Surface Material

One embodiment of the Port may have the external surface of the implanted portion of the Port (16) covered by a material that may produce an ingrowth of well-vascularized tissue and minimal fibrotic tissue. The material may be composed of approved medical-grade silicone and may have a base layer(s) that may be placed on and adhere to the Port's surface and may be of variable thickness. An additional layer of microscopic particles composed of porous silicone of variable size and shape may be adhered to the base layer(s) creating an irregular surface both in height and distribution. (This material and has been submitted to the FDA for PMA approval for human implantation and may be obtained from Healionics Corp. of Seattle, Wash.)

### Seal (FIG. 4, 7)

The Seal in one embodiment may be a cylinder of equal height to the Port and may have a diameter less than the inner diameter of the Port; however, it may be of equal diameter to the Port's inferior rim luminal protrusion (“O” ring) (19) to provide a fluid tight junction whether the Seal is in the nondeployed or deployed position (FIG. 7). The Seal may include a Central Core (24) containing a plurality of Channels and may have a pair of Recesses (21) on opposing sides of the Central Core (24) for a total of four Recesses. Each Recess may have a Seal Lift Tab (23) on its superior medial surface. Each pair of Recesses may have two Lateral Pillars (28) extending between the pair of Recesses and there may be extending from these pillars a Seal Lock Tab (22), i.e., a protrusion, of such shape and dimension as to enable it to sit within the Vertical, Horizontal and Ascending Grooves on the inner surface of the Port. These Seal Lock Tabs and Grooves may control the vertical movement for initiating HD (deployed position) and terminating HD (nondeployed position); and also may control the required sequence of horizontal and ascending vertical movements for removing the Seal and descending vertical and horizontal movements for replacing the Seal.

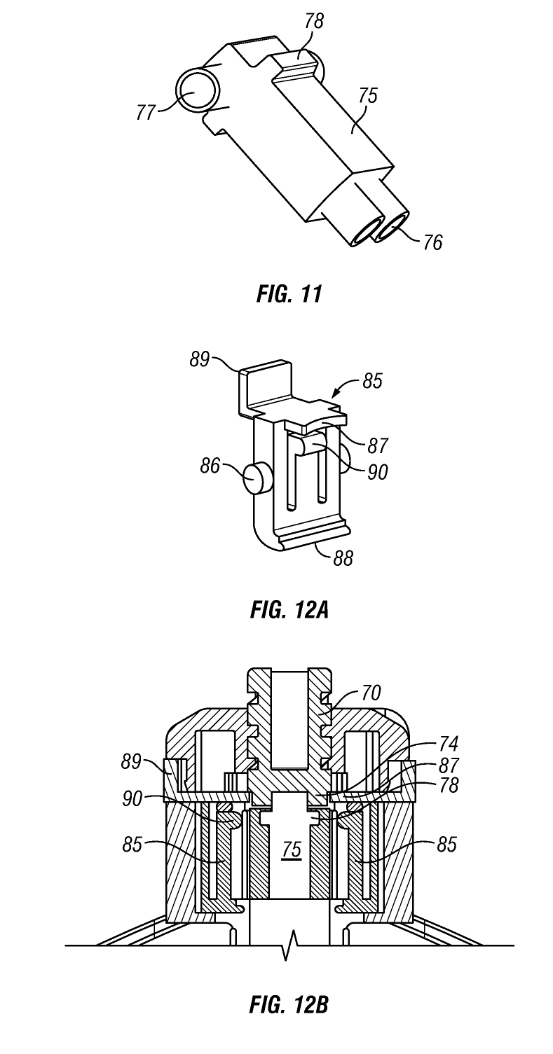

One embodiment of the Seal may contain two parallel Seal channels for transporting blood to and from the Graft lumen, thru the Connector and to the Dialyzer. These channels may be identical in design and located side by side within the Central Core of the Seal (25). The superior orifices of the Seal Channels are present on the superior surface of the Seal. The Upper Section of these Seal Channels (26) may be conical in shape and may decrease in diameter as they proceed inferiorly and may reach a Seal Channel Shelf (27) that may form the superior orifice of the lower section of the channel which may be of smaller diameter than the upper section. The Shelf may act as a “stop” for the Channel Inserter Extensions (76) that may extend from the Channel Inserter (FIG. 11) component of the Connector described below. The wall thickness of the Channel Inserter Extensions may be identical to the width of the Shelf. This configuration of the Upper Section of the Seal Channels may secure the Channel inserter Extensions within the Seal Channels and forms a smooth fluid tight junction between the Channel Inserter Extensions and the Seal Channels during HD. The lower sections of the Seal Channels may be of uniform diameter throughout their length. The Seal Channels may descend for a variable but equal distance before each undergoes an approximate right angle turn producing a pair of Inferior Lateral Orifices (25) on the lateral surfaces of the Seal directed at 180 degrees to each other (25). This results in one Seal Channel orifice directed towards the source of arterial inflow and the second directed towards the venous outflow tract of the Graft when the Seal is deployed in the lumen of the Graft for HD. When the Seal is in both the nondeployed and deployed positions the lateral surface of the Seal abuts the luminal protrusion of the inferior luminal surface of the Port, the “O” ring, to form the fluid tight junction described above. The surface of the Seal superior to this junction may not be in direct contact with the luminal surface of the Port (15) due to the smaller diameter of the Seal as compared to the Port lumen diameter above the site of the “O” ring except at the site, described above, of the Seal Lock Tabs within the Port Grooves. Thus there may be a minimal but finite Space between the surfaces of the Port and Seal to allow fluid of various components to be flushed thru the Seal channels, into this space and exiting the superior orifice of the Port. Flushing may be done at the completion of HD prior to removing the Connector (described below).

### Cap (FIG. 3)

The Port, when not in use, may be covered by a disposable Cap which may be circular and domed shaped with a concave Inner Surface and may extend, when in place, beyond the circumference of the superior surface of the Port Lip. The Cap may lock on to the Port by means of the asymmetrical set of Grooves on the Port's Lip. The locking mechanism is opened by compression of the Cap's Press Tabs (46). Other such mechanisms will also be known. The Cap is then placed on the Port Lip and the Press Tabs released securing the Cap on the Port by means of the Cap Locking Clips (47). The concave Inner Space within the Cap dome may contain a compressible material, i.e., the Cap Antimicrobial Pad (48), that may have sufficient porosity and capacity for absorption and/or adsorption to retain a variable volume of antimicrobial solution of various compositions. When the Cap is placed on the Port at the completion of HD the compression of this material may elute the antimicrobial solution thereby bathing the superior rim of the Port and the superior surface of the Seal with the solution which also may diffuse into the saline solution, which as a result of flushing, is present within the Seal Channels, Recesses and the Space between the Seal and the inner luminal surface of the Port.

The Cap is removed using sterile technique before initiating HD and is replaced by a new sterile Cap using sterile technique at the completion of HD.

### Graft (FIG. 1, 2, 5, 13, 14 Labels 35, 36)

The Graft may be bonded to the Port and joined to an artery and vein and may be composed of PTFE with various configurations, porosities, laminations, and dimensions (wall thickness, diameter, length). Exemplary, but non-limiting, values may be for wall thickness 1.0 mm-2.0 mm, for diameter 5 mm-8 mm, and for length 20 cm-50 cm.

The Graft may be placed deep within the patient's tissues to facilitate implantation of the attached Port, gain access to preferred components of the vascular system, and encourage tissue ingrowth into the Graft's external surface, thereby minimizing the risk of infection.

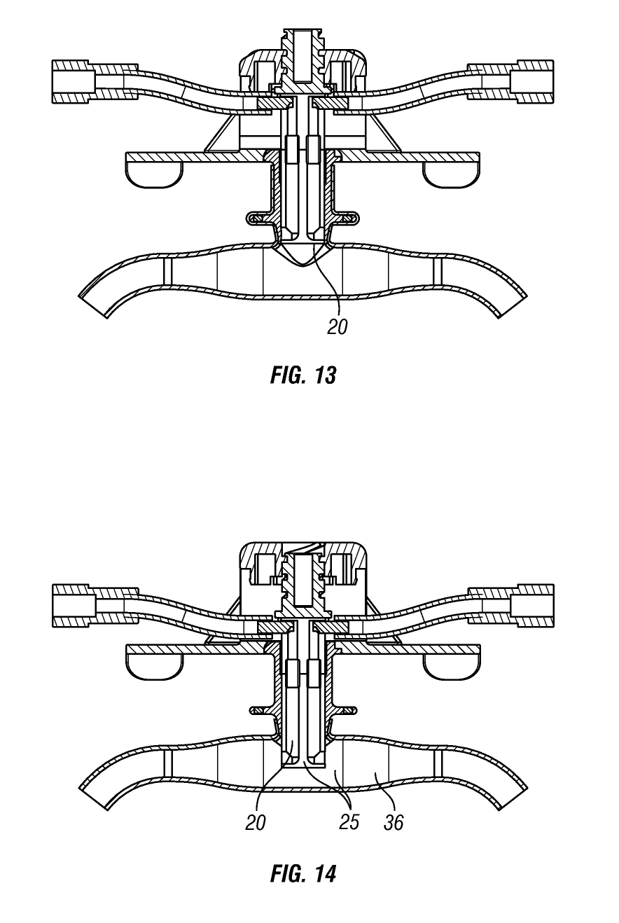

In one embodiment the Graft may be in the form of a tubular Graft with a Right Angled Branch (RAB) (40) of various diameters and lengths. Exemplary, but non-limiting, values may be for diameter 1.25 cm-1.75 cm, and for length 0.75 cm-1.75 cm. The RAB may be positioned at various sites along the graft length but preferably at the section of the Graft having an increased internal diameter (FIG. 5 Label 36). The Graft may be bonded to the Port by means of the RAB that extends from the main Graft lumen. The RAB may be of such a diameter and length as to fit as a “sleeve” (40) on the grooved and roughened external surface of the Port and may extend to the inferior surface of the Flange or may cover the Flange. The “sleeve” of PTFE may have a tight external Wrap of PTFE thread of various dimensions and configurations. The RAB “sleeve” and Wrap may be thermally bonded to the Port to provide a secure attachment. The Port may be positioned within the RAB lumen so that the rim of the Port's inferior orifice may join the main Graft lumen at the origin of the RAB. This configuration may provide a smooth nonthrombogenic interface between the blood flow in the Graft lumen and the junction of the main Graft and the RAB of the Graft, Port rim and Seal when the Seal is in the nondeployed position (FIG. 13).

There may be continuous blood flow thru the main Graft lumen at all times whether or not the system is being used for HD.

The main Graft lumen may be of uniform diameter except for a section extending a variable distance in both directions from the site of the RAB orifice. This section may have an increased internal diameter (36), e.g., of 7 mm-9 mm although these values are not limiting, and which may be sufficiently greater than the diameter of the Seal and the distance the Seal is deployed within the Graft lumen. This enlarged space may prevent the Seal from contact with the Graft internal surface. The Graft diameter may be greatest at the site of the RAB and gradually decrease in both directions until it attains the internal diameter of the main Graft. The increased diameter of this Graft section at the site of Seal deployment may result in blood flow of sufficient volume and velocity to prevent local platelet and fibrin deposition at the PORT-GRAFT interface, and prevent recirculation of dialyzed blood during HD.

The arterial inflow and venous outflow orifices of the Graft may be anastomosed to an artery and a vein using standard suture techniques, or other available means of joining a graft to a blood vessel may be used.

Another preferred embodiment for joining the Graft to the venous outflow tract (VOT) may be the use of a proprietary PTFE lined expandable Stent (e.g., a VasStent™ available from Vas Tech LLC of Los Angeles, Calif.).

The Stent may be placed within the VOT and joined by a tubular extension of various configurations, by various available methods, to the venous end of the Graft. The Graft may also be configured with the Stent as an integral part of the main Graft. In this design the Graft may be continuous with the Stent at a fenestration in the wall of the Stent. The smooth appropriately angled junction of Graft and Stent and the presence of the Stent within the VOT may minimize the development of NIH and VR and prevent VOT stenosis.

### Connector (FIGS. 6, 8, 13, 14)

The Connector when locked on to the Port and activated may enable the Seal and Seal Channel Inferior Lateral Orifices to be deployed within the Graft lumen, providing continuous blood flow from Graft lumen, thru Seal and Connector Channels, and External Blood Tubing (51, 52) to the Dialyzer and the simultaneous return of blood from the Dialyzer to the Graft lumen. At the completion of HD, activation of the Connector may enable the Seal to be returned to the nondeployed position and secured within the Port lumen, removing the Seal Channels from the Graft lumen. The activation of the Connector may enable vertical movements of the Seal and may be readily accomplished by a single maneuver, the manual rotation of a Control Knob (FIG. 9), present on the Connector, and described below.

In one embodiment the Connector may be cylindrical in shape and of variable height, diameter and configuration and may contain the necessary elements so as to enable it to 1) lock on to, in a predetermined orientation, and be released from, an implanted Port's superior rim and Lip and 2) move the Seal a predetermined vertical direction and distance into and out of the Graft lumen (FIGS. 13, 14).

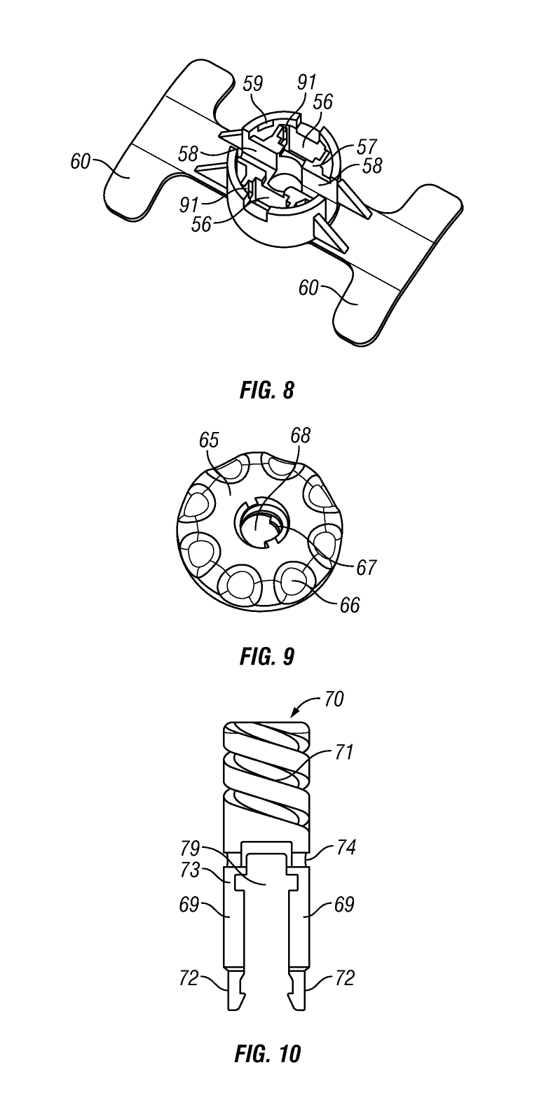

The Body (FIG. 8) of the Connector may include a single cylindrical unit. The Body has single or multiple Recesses (56), Posts (57) and Tracks (58) to configure and maintain the required relationships among the Connector's component parts (FIGS. 9, 10, 11, 12) and to enable and control the Connector's automated maneuvers.

A Stabilizer (60) of variable shape and dimensions may be joined to and extend from the Body and allow the Connector to be secured to the patient's skin to prevent motion of the Port and Connector.

One embodiment of the Stabilizer may be a minimally concave circular Skirt which is an integral part of the Body and which may be reinforced in its attachment to the Body by a variable number of rigid Buttresses (61).

The Control Knob (CK) (FIG. 9) may be of variable dimensions and have the shape of a disc or torus. The periphery of the CK may have a number of equally-spaced shallow indentations for grasping and rotating the CK by means of a circumferential Track on its inferior rim and may snap on to four Tabs (59) on the inner surface of the Connector Body's superior rim. Other numbers of tabs may also be employed. This attaches the CK to the Body of the Connector and allows smooth rotation of the CK in clockwise and counter-clockwise directions without imparting rotation to the Connector Body or the Port, when the connector is locked on to the Port. The CK may have a circular Central Opening (68) lined by a series of Helical Threads (67) of a specified pitch.

The Drive Shaft (DS) (FIG. 10) may be a cylinder with two sections. The upper section may have a series of Screw Threads (71) on its lateral surface, matching those of the Central Opening of the CK, and may be positioned within the Central Opening of the CK thereby meshing the Helical Threads of the CK's Central Opening and the Screw Threads of the DS's upper section. This configuration, in conjunction with a mechanism described below to prevent DS rotation, may only produce either upward or downward vertical movement of the DS depending on the direction of rotation of the CK.

Four opposing, narrow, minimally flexible and elongated Projections (69) of the DS may descend from the cylindrical segment of the DS. Each Projection may have a proximal DS Clamp Release Slot (74), a DS Locking Slot (73) and a distal DS Seal Lifter Hook (72). The DS Locking Slots may be locked on to the Channel Inserter (CI) (FIG. 11) Locking Tabs (78), described below. The locking of the DS on to the CI may prevent the rotation of the DS as the CK is rotated, restricting the DS to vertical movements within the Central Opening of the CK which in turn produce vertical movements of the CI and Seal. The inferior surface of the DS has a recess (79) in which the superior surface of the CI is positioned. These two configurations result in the fixation of the DS to the CI at all times.

The Channel Inserter (CI) may have a pair of parallel channels (25) within it that join the outflow and inflow external blood tubes (51, 52) to the channel inserter extensions (76) extending from the CI's inferior surface. This configuration may provide a continuous succession of conduits that may be of similar or identical diameter throughout their course from seal channels to dialyzer blood tubing junctions. The Tracks on the lateral sides of the Connector Body may allow vertical movement of the external blood tubing in conjunction with the vertical movements of the CI.

The DS may move downward when the CK is rotated clockwise. This movement of the DS may then drive the CI (FIG. 11), to which it is fixed, downward. The downward movement of the DS and CI positions the Channel Inserter Extensions (76) in the upper section of the Seal Channels and at the same time moves the Seal downward, deploying the Seal a FIXED distance into the Graft lumen. The downward movement of the Seal may be limited by the Seal Lock Tabs, within the Vertical Grooves on the inner surface of the Port. When the Lock Tabs reach the lower horizontal edge of the Vertical Groove the downward movement of the Seal is stopped (18V).

The downward movement of the DS may also position the four DS Seal Lifter Hooks (72) beneath the Seal Lift Tabs (23) on the medial surface of the Seal Recesses (21). At the completion of HD, when the CK is rotated counter clockwise, the upward movement of the DS may then retract the Seal upward by means of the DS Seal Lifter Hooks which grasp the DS Seal Lift Tabs (23) and are held in place by the presence of the Port wall as the Seal moves upward within the Port lumen until it reaches the nondeployed position. The upward movement of the Seal may be limited by the Seal Lock Tabs, within the Vertical Grooves on the inner surface of the Port. When the Seal Lock Tabs reach the upper horizontal edge of the Vertical Groove the upward movement of the Seal may be stopped (18V). The upward movement of the DS may also move the Channel Inserter upward because of the fixation of the DS to the CI (79).

When the superior surface of the Seal reaches the superior surface of the Port the relationship of the Seal Lifter Hooks to the Lift Tabs may alter due to 1) the absence of inward compression by the Port wall, 2) the angulation of their opposed surfaces (72), 3) the flexibility of the DS Projections and 4) the action of the Connector Clamp (FIG. 12) and Leaf Spring (90) described below. These factors may allow the Seal Lifter Hooks to slide off the Lift Tabs and release the Seal. As a result of the upward movement of the DS and CI the Channel Inserter Extensions (76) may be removed from the Seal Channels (26). The Connector (FIG. 6) may then be removed from the Port by finger compression to release the Connector Locking Clamps (85) as described below. Finger compression may not release the Connector Locking Clamps (85) until the Seal (FIGS. 4, 7) is secure within the Port lumen and the various Connector component parts allow the Clamp Connector Release Tab (89) to be inserted in the DS Clamp Release Slot (74)

The System may be flushed with a saline solution containing various drugs (anticoagulants and/or antimicrobials) when the Seal is secured within the Port lumen and the Connector is still locked on to the Port and all blood tubing and channels are in continuity. Fluid may be instilled thru both External Blood Tubes (51, 52) using a Y connector and a single syringe or two separate syringes or other available methods. The fluid may flush all Connector component channels and the Seal Channels. The flush solution will exit from the Seal Channel Inferior Lateral Orifices (25) and flush the Spaces between the Seal Surface and the Port luminal Surface (10) and the Seal Recesses (21).

The function of the Connector (FIG. 12A, 12B) lock and release mechanism depends upon the actions and interactions of the Connector Clamps (CC) (FIG. 12A, 12B), the Cut Outs (CO) (91) on the Connector Body (CB) (FIG. 6), the DS and the presence of the asymmetric Port Connector Grooves (17).

The Connector, prior to placement on the Port, may be in the locked position due to the action of the Connector Clamp Leaf Spring (90) which includes a protrusion pushing against the interior core of the Connector Body forcing the Clamp Port Locks (88) inward (medially). In order to unlock the Connector for placement on the Port, the two Clamp Connector Release Tabs (CCRT) (89) present on the opposite sides of the Connector Body (CB) (55) and below the CK (65) may be compressed inward by moderate finger pressure. This action may cause the CCRTs to overcome the inward directed force of the Leaf Spring and rotate on the Clamp Rotation Axle (86). The lower section of the CCRT moves outward (lateral) as the upper section of the CCRT moves inward (medial). This inserts the Clamp-DS Locking Tabs (87) into the DS Clamp Release Slots (74). These actions may move the Clamp-Port Lock (88) outward (laterally) and allow placement of the Connector on the Port. The Asymmetric Configuration of the Clamp-Port Lock may match the Asymmetric Configuration of the Port Connector Grooves in order to seat the Connector on the Port in correct alignment. When the Connector is seated on the Port, the finger pressure on the CCRTs is released. The release of the CCRTs may reverse all of the above actions and interactions. The Clamp Port Locks (88) may be secured within the Port Connector Grooves (17), the Clamp-DS Locking Tab may be removed from the DS Clamp Release Slot (74), and the Connector may be locked on to the Port (FIG. 13). These maneuvers prepare the System to initiate HD as described below in the exemplary Method of Hemodialysis.

### Method of Hemodialysis

Step 1—Remove Cap—Using sterile procedure the Cap is removed from the Port by finger compression of the Cap Press Tabs (46). This releases the Cap Locking Clips (47) and the Cap is removed from the Port and discarded.

Step 2—Lock Connector on Port—Using sterile procedure the locking mechanism of the Connector is opened by finger compression of the Connector Clamp Release Tabs (89). The Connector is then placed in the opened configuration on the superior surface of the Port. The Connector Clamp-Port Lock (88) must match the Port Connector Grooves (17). This match assures proper alignment of the External Blood Tubing and the inflow and outflow blood channels of the Connector and the Seal. The Channel Inserter Extensions (76) extending from the under surface of the Channel Inserter component of the Connector are automatically positioned within the superior orifices of the Seal Channels during this maneuver. Finger compression is then released locking the Connector onto the Port by the action of the Connector Clamp Port Lock.

Step 3—Deploy Seal into Graft lumen—The Control Knob is then manually rotated clockwise which causes the Drive Shaft to move downward forcing the Channel Inserter Extensions into the upper sections of the Seal Channels. The Extensions abut the shelf within the Seal Channels to form a secure fluid tight junction. The downward motion of the Drive Shaft and Channel Inserter (FIG. 11) also moves the Seal downward a fixed distance into the Graft lumen which has an increased diameter in this section of the Graft. This places the Seal Channel openings into the Graft lumen positioned at 180 degrees to each other, one directed towards the arterial inflow and the other directed towards the venous outflow, providing high blood flow to and from the Dialyzer. The Seal occludes, e.g., at most 50% of the increased luminal cross-sectional area of the Graft. The open cross-sectional area allows blood flow to continue thru the Graft lumen during HD. This prevents the recirculation of blood flow returned from the Dialyzer, and also “washes” the Seal and Graft surfaces, preventing deposition of platelets and fibrin.

Step 4—Aspirate Blood—Two External Blood Flow Tubes exit from the Connector and are continuous with the inflow and outflow channels within the Channel Inserter and the Seal. These blood conduits must be aspirated to verify the free flow of blood thru the system, from Graft lumen to External Blood Tubing (FIG. 14), before initiating Dialysis. When this is determined to be satisfactory the Connector blood tubing is joined to the Dialyzer blood tubing and HD is initiated.

Step 5—Initiate Dialysis—The Seal's inflow channel, the Connector's blood flow channel in the Channel Inserter, and the External Blood Tubing transport the patient's arterial blood from the Graft to the Dialyzer, while the dialyzed blood is simultaneously returned from the Dialyzer thru a separate but parallel set of blood tubing and channels to the Graft lumen and directed downstream towards the venous outflow tract of the Graft returning the dialyzed blood to the patient's circulation. Throughout HD substantially no blood enters the Port lumen due to the presence of the titanium “O” ring described above.

Step 6—Terminate Dialysis—At the completion of dialysis the counter clockwise rotation of the Control Knob reverses the Drive Shaft movement and raises the Seal into the Port lumen. At the completion of the upward movement of the Seal into the nondeployed position 1) the Seal is seated within the Port lumen, 2) the Seal and the inferior Port rim present a smooth nonthrombogenic surface to the blood flow within the Graft, and 3) the Drive Shaft has released the Seal. Prior to release and removal of the Connector, the External Blood Tubing, and all Connector and Seal blood channels, the Port lumen and Seal recesses may be flushed with saline containing an anticoagulant substance using the External Blood Tubes as the sites to instill the flush solution.

The Connector to Port locking system is released by finger compression of the Connector Clamp Release Tabs and the Connector is removed. The Connector cannot be released from the Port until the Seal is in the proper position due to the presence of the Drive Shaft Clamp Release Slot. A new sterile Cap containing antimicrobial solution is then locked on to the Port.

All of the described actions to initiate and terminate Dialysis occur within the sterile Body of the Connector, the Port or the Cap and require no manipulation other than 1) finger compression of the locking mechanisms of the Connector or Cap and 2) manual rotation of the Control Knob of the Connector.

The distance and direction of Seal movement within the Port and Graft lumen is controlled and limited, and inadvertent removal of the SEAL when removing the Connector is generally not possible due to the configuration of the grooves on the Port's luminal surface and the Seal Locking Tabs. Removal of the Seal requires the use of a special Seal Tool.

The above description is purely exemplary. It will be understood that a large number of variations of the above may occur and still be within the scope of the invention. For example, the CK may connect to the Connector Body by other than a helical thread system or tabs and tracks. A large number of such variations will be apparent to one of ordinary skill in the art given these teachings.

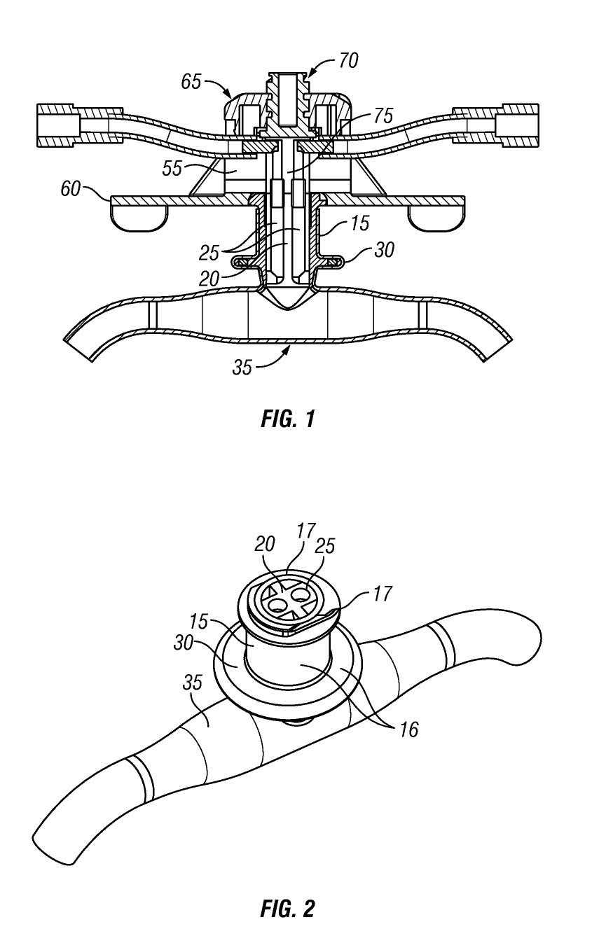

The following list of reference numerals pertains to elements in the drawings:
10 Implant (VasPort)—FIG. 2
14 Port Lip
15 Port—FIG. 7
16 Coating
17 Port Connector Grooves
18 Port Seal Grooves
   18V—Vertical
   18H—Horizontal
   18A—Ascending
19 Port O Ring
Seal—FIG. 4/FIG. 7b
21 Seal Recesses
22 Seal Lock Tab
23 Seal Lift Tabs
24 Seal Central Core
25 Seal Channels
26 Seal Channel Superior Section
27 Seal Channel Shelf
28 Seal Lateral Pillars
30 Port Flange
31 Flange Suture Holes
35 VasPort PTFE Graft
36 Graft Section Increased Diameter
40 Graft Right Angle Branch
45 Cap (VasCap)—FIG. 3
46 Cap Press Tabs
47 Cap Locking Clips
48 Cap Anti-microbial Pad (microbial)
50 Connector (VasConnect)—FIGS. 6
51 & 52 Connector External Blood Flow Tubes (Inflow and outflow)
55 Connector Body—FIG. 8
56 Connector Body Recesses
57 Connector Body Posts
58 Connector Body Tracks (Blood Flow Tubes)
59 Connector Body Control Knob Tab
60 Connector Stabilizer
61 Connector Buttress
62 Connector
65 Connector Control Knob—FIG. 9
66 Control Knob Finger Grip
67 Control Knob Helical Threads
68 Control Knob Central Opening
69 Drive Shaft Projections
70 Connector Drive Shaft—FIG. 10
71 Drive Shaft Screw Threads
72 Drive Shaft Seal Lifter Hooks
73 Drive Shaft Channel Inserter Locking Slot
74 Drive Shaft Clamp Release Slot
75 Channel Inserter—FIG. 11
76 Channel Inserter Extensions
77 Channel Inserter Outflow/Inflow
78 Channel Inserter Locking Tab
79 Drive Shaft/Channel Inserter Recess
85 Connector Port Clamp—FIG. 12A
86 Clamp Rotation Axle
87 Clamp Driveshaft Locking Tab
88 Clamp Port Lock
89 Clamp Connector Release Tab
90 Clamp Leaf Spring
91 Clamp Rotation Axle Cutout

1. A system for vascular access, comprising:
a. a titanium port having an external surface configured to induce tissue ingrowth, the port defining a lumen;
b. a movable seal, positioned within the lumen of the port, to occlude the port, the seal including channels to transport blood, the seal being deployable into a lumen of a vascular graft to provide blood flow during hemodialysis, the seal redeployable into the lumen of the port at a conclusion of hemodialysis;
c. a cap configured to be locked onto and released from the port by a locking mechanism, the cap including an under surface, the under surface including a compressible material configured to absorb and elute a antimicrobial substance.

2. The system of claim 1, wherein the external surface includes a silicone material.

3. The system of claim 1, further comprising a vascular graft, the vascular graft including a section with an increased diameter from which a substantially right angled branch originates and is bonded to the port.

4. The system of claim 3, wherein the vascular graft made of polytetrafluoroethylene.

5. The system of claim 1, further comprising an expandable stent having an angled tubular extension, the extension in fluid communication with the vascular graft.

6. The system of claim 5, wherein the extension is joined to the vascular graft.

7. The system of claim 5, wherein the extension is formed integrally with the vascular graft.

8. The system of claim 5, wherein the expandable stent is covered with polytetrafluoroethylene.

9. The system of claim 1, further comprising a connector configured to lock onto and be released from the port, the connector configured to access the channels within the seal and to control movements of the seal within the lumen so as to deploy the channels within the graft lumen and to retract the seal to its nondeployed position within the Port lumen.

10. The system of claim 1, further comprising a seal tool, configured to remove the seal from the port lumen and to replace the seal with a new seal from within a housing of the seal tool.

11. The system of claim 1, wherein the external surface includes a coating to reduce an incidence of local infection.

12. The system of claim 1, wherein the port further comprises an exposed lip that provides an asymmetric locking mechanism that, when interfaced with a connector locking mechanism, alliance channels in the connector with the seal channels in a configuration that conducts blood flow to inflow and outflow tubing of a dialyzer.

13. The system of claim 1, wherein the port further comprises a luminal surface that defines a set of routes that, in conjunction with the seal, provide a method of controlling movements of the seal and securing the seal within the port lumen.

14. The system of claim 13, wherein the luminal surface is further configured to a fixation of at least one drive shaft lifter book to a seal lift tab four providing upward movement of the seal into the port lumen.

15. The system of claim 1, wherein the port has a configuration at an inferior luminal orifice that provides a blood tight junction with the seal to prevent blood from entering the port lumen.

16. The system of claim 1, wherein the port has, in a nondeployed position, a configuration that presents, in conjunction with the seal and the graft, a smooth non-thrombin to surface to a flow of blood within the graft.

17. The system of claim 1, wherein the port further comprises a perforated flange extending from the external surface of the port and which provides for affix eight and of the port to deep tissues and for stabilizing the port for immediate use of the port following implantation.

18. The system of claim 1, wherein the graft section of increased diameter include sufficient cross-sectional area to allow deployment of the seal within a graft lumen, prevent contact between the seal and the graft, or provide high blood flow volumes for hemodialysis, prevention of thrombus formation, and prevention of recirculation of blood returned from a dialyzer.

19. A method of performing hemodialysis, comprising:
a. removing a cap from a port, the port installed in a patient and providing access to a blood flow therein;
b. locking a connector onto the port, the connector providing inflow and outflow lines to a dialyzer;
c. deploying a seal into a graft lumen, the graft lumen defined within a attached to the port;
d. aspirating blood using the inflow and outflow lines and inflow and outflow channels within the seal;
e. initiating dialysis using the inflow outflow lines and inflow and outflow channels within the seal;
f. terminating dialysis;
g. raising the seal into the port lumen; and
h. locking a new sterile cap onto the port.

20. A connector, comprising:
a. a substantially cylindrical housing, including:
i. means for locking on to an implanted port; and
ii. means for moving a seal into and out of the graft lumen.

21. The connector of claim 20, wherein the moving means includes a control knob.
